(19) United States
(12) Patent Application Publication
(10) Pub. No.: US 2022/0340630 A1
(43) Pub. Date: Oct. 27, 2022

(54) COMPOSITION OF RENATURATION BUFFER SOLUTION FOR DIMERIC PROTEINS AND METHOD OF RENATURATION DIMERIC PROTEINS USING THE COMPOSITION THEREOF

(71) Applicant: NATIONAL SCIENCE AND TECHNOLOGY DEVELOPMENT AGENCY, Pathumthani (TH)

(72) Inventors: Katanchalee MAI-NGAM, Pathumthani (TH); Satrawut CHAROENLA, Pathumthani (TH); Warobon NOPPAKUNMONGKOLCHAI, Pathumthani (TH); Ubolsree LEARTSAKULPANICH, Pathumthani (TH); Rachaneegorn GESORN, Pathumthani (TH)

(73) Assignee: NATIONAL SCIENCE AND TECHNOLOGY DEVELOPMENT AGENCY, Pathumthani (TH)

(21) Appl. No.: 17/642,422

(22) PCT Filed: Jul. 17, 2020
(86) PCT No.: PCT/TH2020/000048

§ 371 Date: Mar. 11, 2022

(30) Foreign Application Priority Data
Sep. 13, 2019 (TH) 1901005657
Sep. 13, 2019 (TH) 1901005658

(51) Int. Cl. C07K 14/495; C07K 1/02; C07K 1/22

(52) U.S. Cl. CPC C07K 14/495; C07K 1/02; C07K 1/22

(57) ABSTRACT

A composition of renaturation buffer solution for dimeric proteins and method for using it. The renaturation buffer solution comprises buffer, oxido shuffling system, primary additives, chelating agent and polysorbate compound. The method for renaturation of dimeric proteins includes solubilization of target proteins using a solubilization buffer, solution dilution of the renatured target protein using a renaturation buffer containing polysorbate compound(s), concentrating of the target protein solution to a concentration approaching saturation. A method of applying renature denatured or misfolded proteins to become correctly folded and bioactive. A method of proper renaturation of recombinant proteins that have been expressed using translation vehicles (e.g., bacteria, insects, etc.) and proteins damaged by mechanical shearing, chemical stresses, and other stresses.

### TECHNICAL FIELD

This invention relates to biotechnology in composition of renaturation buffer solution for dimeric proteins and method of renaturation dimeric proteins using the composition thereof.

### BACKGROUND ART

A reducing environment of bacterial cytosol generally inhibits disulfide bonds formation of multiple cysteine containing proteins, leading to misfolding and inclusion body expression. In order to succeed in in-vitro renaturation of multiple cysteine containing proteins in inclusion body form, polypeptide chains have to be not only properly folded into correct secondary, tertiary and quaternary structures, but intra- and/or inter-chain disulfide bond formation at the correct positions is also required. For example, bone morphogenetic protein-2 (BMP-2) is a biologically active homodimer having a disulfide bond between the two monomers. Each BMP-2 monomer contains 7 cysteine residues, forming 3 intra-chain disulfide bonds and a single inter-chain bond.

Metal ion catalyzed air oxidation can promote disulfide bond formation of proteins with low reaction rate and yield (Ahmed A K, et al., J Biol Chem. 1975; 250: 8477-8482). However, chemical-induced oxidation of cysteine residues randomly and non-specifically occurs. Formation of erroneous disulfide bonds and incorrectly folded isomers, as a consequence, requires more complicating purification and possibly leads to reduction or loss of protein activity.

The more common and effective method is the use of oxido shuffling systems, consisting of an oxidizing and a reducing sulfhydryl component. The oxido shuffling systems promote initial oxidation of free sulfhydryl groups and disulfide rearrangement, resulting in a greater extent of the correct disulfide bond formation. The mostly frequently used system is a mixture of reduced (GSH) and oxidized (GSSG) glutathione, referred to as a GSH/GSSG system. However, other low molecular weight thiols, e.g. cysteine/cystine, cysteamine/cystamine, 2-mercaptoethanol/2-hydroxyethyl disulfide can also successfully used, depending on the refolding target proteins (Rudolph R, et al., FASEB J. 1996; 10: 49-56). The prior art of Nasrabadi et al. reported that the efficiency of the cysteine/cystine system in BMP-2 renaturation was comparable to that of the GSH/GSSG system (Nasrabadi D, et al., Avicenna J Med Biotechnol. 2018 October-December; 10(4): 202-207). Moreover, most prior arts coadded 2-5 mM ethylenediaminetetraacetic acid (EDTA) together with the oxido shuffling agents to suppress the oxygen induced oxidation of thiol groups.

Various conventional methods for renaturation of transforming growth factor-β (TGF-β) superfamily members, which are cystine-knot dimeric proteins, have been disclosed. The conventional renaturation methods can be generally divided into 2 steps: solubilization and renaturation. The first is to solubilize denatured proteins in a solubilization buffer and the latter is to simply perform rapid dilution renaturation of the target protein by exposing to an oxido-shuffling refolding buffer. The reducing agents are generally used at concentrations in the range of 1 to 10 mM and the ratios of reducing to oxidizing agents of 10:1 to 1:3 are preferred. (Gieseler G M, et al., Appl Microbiol Biotechnol. 2017; 101: 123-130; Gieseler G M, et al., Biotechnol Rep (Amst). 2018; 18:e00249; Groppe J, et al., J Biol Chem. 1998; 273:29052-29065; Hillger F, et al., J Biol Chem. 2005; 280: 14974-14980; Kuo M M, et al., Microb Cell Fact. 2014; 13:29; Nasrabadi D, et al., Avicenna J Med Biotechnol. 2018; 10: 202-207; Vallejo L F, et al., J Biotechnol. 2002; 94: 185-194; Vallejo L F, et al., Biotechnol Bioeng. 2004; 85: 601-609; U.S. Pat. Nos. 5,756,308; 6,057,430; 6,596,511; 7,354,901 B2)

Most TGF-β proteins contain multiple hydrophobic patches on their surface and prone to self-aggregate in high concentration solutions. Because aggregation is a higher order process than disulfide bond formation, rapid dilution renaturation of TGF-β have been reported to succeed at a fixed protein concentration in the range of 0.05-0.4 mg/mL. Vallejo L F and coworkers have reported optimal refolding concentrations of less than 0.3 mg/mL to achieve the highest renaturation yield of BMP-2 with low aggregation formation. (Vallejo L F, et al., Biotechnol Bioeng. 2004; 85: 601-609). The renaturation yield is expressed as a percentage of dimerized target protein compared to the total target protein having both monomer and dimer forms. Note that oligomer larger than dimer and conformational differences of dimer are not taken into account to the calculation.

Addition of additives to renaturation buffers can enhance renaturation ability of proteins. Various types of commonly used additives include amino acid (such as arginine, cysteine and proline), surfactants (such as 2-4-(2 4 4-trimethylpentan-2-yl)phenoxy, polysorbate 20 and 3-[(3-cholamidopropyl)-dimethylammonio]-1-propane sulfonate), salts (such as sodium chloride), osmolytes (such as sucrose, glycerol and sorbitol), polymers (such as polyethylene glycol and polyvinyl pyrrolidone) and chaotrope (such as urea and guanidinium chloride). Generally, these additives can be divided based on their functions into 2 major groups: folding enhancer and aggregate suppressor.

A surfactant which is commonly used as a primary additive for renaturation of TGF-β superfamily is 3-[(3-cholamidopropyl)dimethylammonio]-1-propanesulfonate (CHAPS). CHAPS is a zwitterionic surfactant with a ring steroid-type chemical structure (Li Z, J Orthop Res. 2017; 35: 51-60) and widely used folding enhancer. However, mechanism of CHAPS as a dimerization enhancer for renaturation of TGF-β superfamily is not known (Honda J, et al., J Biosci Bioeng. 2000; 89: 582-589). U.S. Pat. No. 6,084,076A described a refolding condition suitable for activin A, a member of TGF-β superfamily, which employs surfactant and non-surfactant, charged and non-charged ring steroid-type compounds as effective dimerization enhancers. These include 3-(3-cholamidopropyl)-dimethylammonio-2-hydroxy-1-propane sulfonate (CHAPSO), cholic acid, deoxycholic acid, taurocholic acid, taurodeoxycholic acid and digitonin.

The effective concentration of the CHAPS for renaturation of TGF-β superfamily is moderately high, about 2%-4%. CHAPS, due to its high critical micelle concentration (CMC), is removable from the protein solution, but it is, especially in industrial scale, very costly. Therefore, some prior arts, for example Gieseler et al. 2017, Vallejo et al. 2004, Vallejo et al. 2002 (Gieseler G M, et al., Appl Microbiol Biotechnol. 2017; 101: 123-130, Vallejo L F, et al., Biotechnol Bioeng. 2004; 85: 601-609, Vallejo L F, et al., J Biotechnol. 2002; 94: 185-194) disclosed the use of zwitterionic additives with six-membered ring structure, two hundred-fold less expensive than CHAPS, as primary additives for renaturation of the TGF-β superfamily. These include 2-(cyclohexylamino)ethanesulfonic acid (CHES), 3-(1-pyridinio)-1-propanesulfonate, pyridine-3-sulfonic acid and nicotinic acid. Based on the literature review, there are no comparative studies on efficiency of CHAPS and CHES on the renaturation of the TGF-β superfamily proteins.

Arginine has been widely employed as an aggregate suppressor and a folding additive for longer than 3 decades (Lange C, et al., Curr Pharm Biotechnol. 2009; 10: 408-414). There are a number of prior arts that described of using arginine acid that costs about half the price of CHES, as the primary additive for the renaturation of the TGF-β superfamily proteins. Some, for example Vallejo 2004, Nasrabadi 2018, Hillger 2005, von Einem 2010 and U.S. Pat. No. 7,354,9012, has arginine as a primary additive alone (Vallejo L F, et al., Biotechnol Bioeng. 2004; 85: 601-609, Nasrabadi D, et al., Avicenna J Med Biotechnol. 2018; 10: 202-207, Hillger F, et al., J Biol Chem. 2005; 280: 14974-14980, von Einem S, et al., Protein Expr Purif. 2010; 73: 65-69). Co-use of arginine with CHAPS have been reported in U.S. Pat. No. 5,756,308A and some other literatures (Honda J, et al., J Biosci Bioeng. 2000; 89: 582-589, Gieseler G M, et al., Biotechnol Rep (Amst). However, Kuo and his colleagues found that co-addition of arginine with CHAPS unwantedly decreased renaturation yield of BMP-9 and data on utilizing arginine alone were not reported. Note that the renaturation yield is calculated as a percentage of the amount of dimeric target protein as compared to total target proteins (Kuo M M, et al., Microb Cell Fact. 2014; 13:29). A directly comparative study of the effect of arginine and CHES on the renaturation process of BMP-2 was reported by Vallejo and coworkers (Vallejo L F, et al., Biotechnol Bioeng. 2004; 85: 601-609). Arginine, the most common aggregation suppressor, was more efficient in preventing aggregation of rhBMP-2 during refolding, but CHES was superior with respect to the final renaturation yield of 43%, as compared to that of 15%-30% for the arginine after a 3-day refolding time. In order to obtain a renaturation yield of 50%, Hillger and his coworkers spent 14 days to renature the BMP-2 using arginine acid as a primary additive (Hillger F, et al., J Biol Chem. 2005; 280: 14974-14980).

Dimerization via disulfide bond formation theoretically occurs faster as increasing the protein concentration in the renaturation buffer, due to that the molecules have more opportunity to approach one another. However, in practice, dimerization is hampered by a higher order process of aggregation driven by attractive forces between surface hydrophobic patches. Vallejo and coworkers have reported that, concomitant to low aggregation, a high renaturation yield was observed at a refolding BMP-2 concentration of 0.3 mg/mL (Vallejo L F, et al., Biotechnol Bioeng. 2004; 85: 601-609) Therefore, most prior arts have performed the renaturation of the TGF-β superfamily proteins under very low protein concentrations and consequentially consuming a long period of time to gain a high renaturation yield, especially for using arginine as a primary additive.

von Einem and his colleagues have developed a regeneration method for BMP-2 using arginine as a dimerization agent (von Einem S, et al., Protein Expr Purif. 2010; 73: 65-69). They speculated that the formation of the intermolecular disulfide bond between two approaching BMP-2 monomers would constitute the rate limiting step. The renaturation can be divided into 2 steps. The first step was refolding under a mild condition, i.e., low solution concentrations of the protein and oxido-shuffling system, to allow a slow formation of the intermolecular disulfide bridge. This provides a sufficient time for formation of the intramolecular cystine knot, resulting into a correctly folded BMP-2 monomer (Nasrabadi D, et al., Avicenna J Med Biotechnol. 2018; 10: 202-207). After increasing the protein concentration to allow BMP2 monomers to get closer and removal of oxido-shuffling system for a total of 18 hours, a renaturation yield of only 40%-50% was estimated. The second step was to stimulate disulfide formation between two approaching monomers to obtain a biologically active dimer. The renaturation buffer in this step contained a high concentration of the oxidizing agent to induce interchain disulfide formation, leading to a high renaturation yield of 70%-80% after 3 days. The sodium dodecyl sulfate polyacrylamide gel electrophoresis (SDS-PAGE) analysis of the final BMP-2 product showed two clearly separated bands with similar intensity of both BMP-2 monomer and dimer, suggesting misfolding and/or incorrect dimerization at about 50%. However, the percentages of renaturation yield reported in the prior art were evaluated using combining amounts of both correct and incorrect folded protein. Moreover, the renaturation method reported by von Einem and his colleagues utilized renaturation buffers with different concentrations of the oxido-shuffling system in each step. Therefore, dialysis method against buffered solutions containing the costly arginine is required to exchange media in two different steps, leading to double the used amount of arginine as compare to the conventional method. The renatured BMP-2 was purified using heparin sepharose column.

Addition of secondary additives, such as sodium chloride, urea, guanidine hydrochloride, glycerol, proline and glycine, is another technique that is commonly used to improve the renaturation yield of the TGF-β superfamily proteins. However, only some prior arts have comparatively studied the effects of each secondary additive on protein refolding. The results vary depending on various factors, such as the type of target protein, and the type and concentration of primary and secondary additives. As an example, Vallejo and his coworkers have reported that the use of guanidine hydrochloride as a secondary additive at a concentration of 0.25-0.75 M could reduce aggregation and enhance renaturation yield of BMP-2 when using CHES as a primary additive. But on the other hand, increasing the concentrations to greater than 0.75 M resulted in increased aggregation and reduced renaturation yield. Sodium chloride reduced the BMP-2 aggregation when using CHAPS as a primary additive with a concentration less than 0.5 M (Vallejo L F, et al., Biotechnol Bioeng. 2004; 85: 601-609. In addition, Kuo and his colleagues (2014) described that, for the renaturation system of BMP-9 with CHAPS as a primary additive, secondary addition of urea, guanidine hydrochloride and proline resulted in reduced renaturation yields, while adding glycerol slightly increased the renaturation yield (Kuo M M, et al., Microb Cell Fact. 2014; 13:29).

U.S. Pat. No. 6,057,430 described production of transforming growth factor beta-3, TGF-β-3) using oxido shuffling refolding systems that contain surfactants with a ring steroid-type structure, including digitonin, 3-[(3-cholamidopropyl) dimethylammonio]-1-propanesulfonate (CHAPS) and 3-(3-cholamidopropyl) dimethylammonio-2-hydroxy-1-propane sulfonate (CHAPSO) and a mixture of CHAPS and CHAPSO. In which the inventors used organic solvent additives, such as dimethyl sulfoxide (DMSO), dimethyl sulfone (DMSO₂) and dimethylformamide (DMF), to specifically and efficiently promote dimerization. Moreover, salts and arginine amino acid were also added to the renaturation buffer.

From the literature search, there are no any prior arts about the comparative effects of secondary additives in the renaturation in TGF-β superfamily using arginine as a primary additive at both laboratory and industrial scales. A screen test performing in 1.5 mL test tubes reported that addition of guanidine hydrochloride at a concentration of 550 mM increase the renaturation yield of BMP-2 and a better result was obtained when added with magnesium chloride and calcium chloride, each at a concentration of 2.2 mM. On the other hand, adding guanidine hydrochloride together with sodium chloride and potassium chloride at a total concentration of about 300 mM and sucrose at a concentration of 440 mM caused a decrease of BMP-2 renaturation to form dimer. Polyethylene glycol, ethylenediaminetetraacetic acid and lauryl maltose exhibited no influence on the BMP-2 renaturation using arginine as a primary additive (Shinong Long S, et al., Protein Expr Purif. 2006; 46: 374-378).

Polysorbates or Tweens are nonionic surfactants of ethoxylated sorbitan esters, composing a hydrophilic polyethylene glycol head group and a hydrophobic alkyl tail. They are widely use in protein formulation to prevent interface-induced protein aggregation. There are different types of polysorbate, including polysorbate 20 (tween 20), polysorbate 40 (tween 40), polysorbate 60 (tween 60), polysorbate 80 (tween 80) and polysorbate 85 (polysorbate 85). Polysorbate 20 and polysorbate 80 are the most widely used. Polysorbates are low price surfactants that are safe for food and pharmaceutical industries, and have no impact on protein activities. Both polysorbate 20 and polysorbate 80 can be used as aggregate suppressors in the protein renaturation buffers for aggregation-prone denatured state (Ho J G, et al., Biotechnol Bioeng. 2004; 87: 584-592; Kötzler M P, et al., Protein Sci. 2017; 26: 1555-1563) and aggregation-prone folding intermediate (Pan J C, et al., Biochem Cell Biol. 2005; 83:140-146). However, aggregation suppression efficiency of polysorbate 20 and polysorbate 80, in some cases, is at a limited level, as found that insoluble protein aggregate could only dissociate into non-bioactive soluble oligomer with no disulfide bond formation (Lee S H, et al., Protein Sci. 2006; 15: 304-313; Esmaili I, et al., Res Pharm Sci. 2018 October; 13(5):413-421).

U.S. Pat. No. 6,084,076A described a study of optimum conditions for renaturation of activin A that belongs to the TGF-β family. It found that polysorbate 20 and polysorbate 80 addition as secondary additives does not enhance the activin A renaturation using CHAPS as a primary additive and the bioactive activin A homodimer was not obtained. Based on the literature search, there are no prior arts about using polysorbate as an additive in renaturation buffers for BMP-2 or other proteins in the TGF-β family. One main reason that polysorbates are not commonly used as buffer additives for protein renaturation is their removal difficulty from the protein solutions and their limited use only at a low concentration range. Polysorbates are composed of large fatty acid; therefore, large micelles with low critical micelle concentration are formed.

China Patent No. 103626834A relates to a protein renaturation buffer solution, having tween-20 as a component, at a concentration of 1%, for protein renaturation with the protein concentration greater than 1 mg/mL. An example in the patent described about a member of TGF-β superfamily, inhibin, which is active in two dimeric forms, inhibin-A and inhibin-B, of different inhibin subunits. However, the patent protected renaturation buffer solution is specifically used only in the refolding of the monomeric inhibin subunits to obtain about 90% yield, but not the dimerization between the subunits of inhibin-A and inhibin-B, to resume their natural forms.

### SUMMARY OF THE INVENTION

This invention relates to a composition of renaturation buffer solution for dimeric proteins and method for using it. The purpose of this invention is to reactivate the denatured protein especially biologically active dimeric cystein-rich proteins. The renaturation buffer solution consists of buffers, oxido shuffling system, primary additives, chelating agents and polysorbate compounds. The method for renaturation of dimeric proteins of this invention includes solubilization of target proteins using a solubilization buffer, solution dilution of the renatured target protein using a renaturation buffer containing polysorbate compound, concentrating of the target protein solution to a concentration approaching saturation. This invention can be applied to renature denatured or misfolded proteins to become correctly folded and bioactive. Moreover, it can be used, but not limited, for proper renaturation of recombinant proteins that have been expressed using translation vehicles (e.g., bacteria, insects, etc.) and proteins damaged by mechanical shearing, chemical stresses, and other stresses.

### BRIEF DESCRIPTION OF DRAWING

FIG. 1 shows an SDS-PAGE analysis of recombinant BMP-2 protein obtained from the screen test after 24 hours renaturation using the renaturation buffers for dimeric proteins of this invention. Lane M: Marker; Lanes 1: Renaturation using the buffer solution of this invention without an oxido shuffling system and a polysorbate compound; Lanes 2-6: Renaturation using the buffer solution of this invention with polysorbate 80 at a concentration of 0.01%, 0.025%, 0.05%, 0.075% and 0.1%, respectively; Lanes 7-9: Renaturation using the buffer solution of this invention with polysorbate 20 at a concentration of 0.01%, 0.05% and 0.1%, respectively; Lanes 10: Renaturation using the buffer solution of this invention without a polysorbate compound.

FIG. 2 shows an SDS-PAGE analysis of recombinant BMP-2 protein obtained from the method of the present invention and the prior art (von Einem S, et al., Protein Expr Purif. 2010; 73: 65-69) after increasing protein concentration approaching saturation and stirring for different periods of time. Lane M: Marker; Lanes 1 and 2: Renaturation using the prior art method for 0 and 48 hours, respectively; Lanes 3-5: Renaturation using the method of the present invention with 0.05% polysorbate 80 for 0, 24 and 48 hours, respectively.

FIG. 3 shows purification via heparin affinity chromatography of recombinant BMP-2 produced using the renaturation method of the present method with 0.05% polysorbate 80. Lane M: Marker; Lane L: Loaded protein; Lane FT: Flow through protein; Lane W1 and W2: Washed fractions at sodium chloride concentrations of 0.3 and 0.4 M, respectively; Lane number corresponds to fraction number eluted at different concentrations of sodium chloride.

FIG. 4 shows purification via size exclusion chromatography of recombinant BMP-2 produced using the renaturation method of the present method with 0.05% polysorbate 80. Lane M: Marker; Lane number corresponds to fraction number.

FIG. 5 shows purification via heparin affinity chromatography of recombinant BMP-2 produced using the renaturation method of the prior art (von Einem S, et al., Protein Expr Purif. 2010; 73: 65-69). Lane M: Marker; Lane L: Loaded protein; Lane FT: Flow through protein; Lane W: Washed fractions; Lane number corresponds to eluted fraction number.

FIG. 6 shows purification via size exclusion chromatography of recombinant BMP-2 produced using the renaturation method of the prior art (von Einem S, et al., Protein Expr Purif. 2010; 73: 65-69). Lane M: Marker; Lane number corresponds to fraction number.

FIG. 7 shows alkaline phosphatase activity of C2Cl2 cell lines cultured in the presence of rhBMP-2 purified using size exclusion chromatography. The tested proteins were renaturated using the renaturation buffers with and without polysorbate as a secondary additive.

FIG. 8 shows alkaline phosphatase activity of C2Cl2 cell lines cultured in the presence of rhBMP-2 after purified using size exclusion chromatography. The tested proteins were produces using the method of the present invention and the prior art (von Einem S, et al., Protein Expr Purif. 2010; 73: 65-69).

### DISCLOSURE OF INVENTION

The present invention relates to a composition of renaturation buffer solution for dimeric proteins, especially biologically active dimeric cystein-rich proteins, and method for using it. This invention can be applied to renature denatured or misfolded proteins to become correctly folded and bioactive. Moreover, it can be used, but not limited, for proper renaturation of recombinant proteins that have been expressed using translation vehicles (e.g., bacteria, insects, etc.) and proteins damaged by mechanical shearing, chemical stresses, and other stresses.

The renaturation buffers of the invention use polysorbate as a secondary additive to synergistically enhance performance of primary additives. The primary additives include oxido shuffling systems, folding enhancers and aggregate suppressors. The synergistic effect is to quantitatively and qualitatively promote dimerization, e.g. having a greater rate and possessing a more bioactive conformation, with no increase in redox system concentration and no impact on purification ability of the proteins. This benefits in reducing production cost and cycle time.

A composition of renaturation buffer solutions for forming dimeric, biologically active proteins comprises:
a) 20-250 mM buffer at pH range of 6-10,
b) Oxido shuffling system at a concentration of 0.05-5 mM and molar ratios of reduced and oxidized thiol of 20:1 to 1:20,
c) 0.1-5 M primary additive,
d) 1-10 mM chelating agent,
e) 0.02-0.2% V polysorbate compound.

The composition of renaturation buffer solutions for forming dimeric, biologically active proteins further comprises secondary additives.

An oxido shuffling system is a mixture of reducing and oxidizing thiol.

Additives of the present invention can be divided into 2 groups: primary additive and secondary additive. The primary additive is referred to as the ones that can promote protein renaturation on the own, without the addition of other additives. Additives stimulating protein dimerization are not defined as the primary additives. The secondary additive is referred to as the ones that need to be combined with primary additive to promote protein renaturation.

A method for renaturation of dimeric proteins of the present invention comprises the following steps:
a) dissolving a denatured protein in a solubilization buffer solution to a final concentration of 20-60 mg/mL,
b) diluting the denatured protein solution from a) using a renaturation buffer solution containing a polysorbate compound at a temperature of 0-25° C. for 1-24 hours to a final concentration of 0.05-0.75 mg/mL. The said renaturation buffer solution consists of 20-250 mM buffer at a pH range of 6-10, a 20:1 to 1:20 mixture of reducing and oxidizing thiol at a concentration of 0.01-5 mM, 0.1-5 M primary additive and 0.02-0.2% V polysorbate compound,
c) concentrating the protein solution obtained from b) to a concentration approaching saturation and stirring at a temperature of 0-25° C. for 1-48 hours.

The method for renaturation of dimeric proteins further comprises steps of purifying the protein using heparin affinity and size exclusion chromatography.

The dissolving a denatured protein is carried out in a solubilization buffer solution to a final protein concentration of 1-20 mg/mL at a temperature of 0-30° C. for 1-48 hours. The pH of the protein solution is then adjusted to a range of 3-4 and the reducing agent is completely removed using dialysis. The protein concentration is increased to a final concentration of 20-60 mg/mL. The preferred condition for the dissolving step is at a temperature of 25° C. for 2 hours.

A solubilization buffer solution comprises:
a) 20-250 mM buffer at pH range of 2-12,
b) 5-200 mM denaturant,
c) 5-200 mM reducing agent for disulfide bond formation.

The composition of the solubilization buffer solution further comprises chelating agent, aggregate suppressor, and mixture thereof.

The preferred condition for the diluting step is at a temperature of 4° C. for 24 hours.

The renaturation buffer solution containing a polysorbate compound consists of buffer solution, a mixture of reducing and oxidizing thiol, primary additives and a polysorbate compound. The renaturation buffer solution further comprises a chelating agent at a concentration of 1-10 mM.

The preferred composition of the renaturation buffer solution containing a polysorbate compound comprises:
a) 100 mM buffer at pH range of 8-9,
b) a mixture of reducing and oxidizing thiol at a concentration of 0.05-1 mM and a molar ratio of reduced and oxidized thiol of 10:1 to 1:10,
c) 0.25-3 M primary additives,
d) 0.05% V polysorbate compound,
e) 5 mM chelating agent.

The preferred condition for concentrating the protein solution is at a temperature of 4° C. for 48 hours. The concentrating step is performed using ultrafiltration technique that is selected from crossflow filtration technique, centrifugal filter device, and combination thereof.

Additional details of solubilization buffer solution and renaturation buffer solution containing a polysorbate compound are as follows:

The buffer is selected from Good's buffers.

The preferred Good's buffer is selected from the group consisting of Tris buffer, HEPES buffer, phosphate buffer, MES buffer, tricine buffer, and mixture thereof. The preferred buffer is Tris buffer. The preferred concentration of buffer is 100 mM and the preferred pH is 8-9.

The oxido shuffling system, or mixture of reducing and oxidizing thiol compounds, is selected from the group consisting of a reduced glutathione (GSH)/oxidized glutathione (GSSG) mixture, a cysteine/cystine mixture, a cysteamine/cystamine mixture, a 2-mercaptoethanol/2-hydroxyethyl disulfide mixture, and mixture thereof. The preferred mixture of reducing and oxidizing thiol is a reduced glutathione and oxidized glutathione mixture or a cysteine and cystine mixture.

The primary additive of the present invention acts to enhance protein folding or suppress protein aggregation. The primary additive is selected from the group consisting of arginine, glutamate, 2-(cyclohexylamino)ethanesulfonic acid (CHES), 3-(1-pyridinio)-1-propanesulfonate, pyridine-3-sulfonic acid, nicotinic acid, 3-[(3-cholamidopropyl)-dimethylammonio]-1-propane sulfonate (CHAPS), 3-(3-cholamidopropyl) dimethylammonio-2-hydroxy-1-propane sulfonate (CHAPSO), cholic acid, deoxycholic acid, taurocholic acid, taurodeoxycholic acid, digitonin, and mixture thereof. The preferred primary additive is arginine. The preferred concentration of primary additive is 0.25-3 M.

The chelating agent is selected from the group consisting of ethylenediaminetetraacetic acid (EDTA), pentetic acid or diethylenetriaminepentaacetic acid (DTPA), N-(2-hydroxyethyl)ethylenediaminetriacetic acid (HEDTA), egtazic acid or ethylenebis(oxyethylenenitrilo)tetraacetic acid (EGTA), nitrilotriacetic acid (NTA), dimercaptosuccinic acid (DMSA), 2,3-dimercapto-1-propanol (DMP), penicillamine, and mixture thereof. The preferred chelating agent is ethylenediaminetetraacetic acid. The preferred concentration of the chelating agent is 5 mM.

The polysorbate compound is selected from the group consisting of polysorbate 20, polysorbate 40, polysorbate 60, polysorbate 80, polysorbate 85, and mixture thereof. The preferred polysorbate compound is polysorbate 80. The preferred concentration of the polysorbate compounds is 0.05-0.1% V.

The reducing agent for disulfide bond formation is selected from the group consisting of dithiothreitol, dithioerythritol, 2-mercaptoethanol, tris(2-carboxyethyl)phosphine, reduced glutathione, and mixture thereof. The preferred reducing agents is dithiothreitol.

The secondary additive is selected from the group consisting of denaturant, amino acid, salt, sugar, alcohol compound, surfactant, and mixture thereof. These secondary additives synergistically enhance protein folding or reduce protein aggregation.

The denaturant is selected from the group consisting of guanidine hydrochloride, urea, thiourea, 2-mercaptoethanol, dithiothreitol, tris(2-carboxyethyl)phosphine, and mixture thereof. The preferred denaturant is guanidine hydrochloride.

The amino acid is selected from the group consisting of proline, glycine, arginine, lysine, alanine, leucine, isoleucine, valine, phenylalanine, tyrosine, tryptophan, asparagine, glutamic acid, glutamine, aspartic acid, serine, threonine, cysteine, methionine, histidine, and mixture thereof.

The salt is selected from the group consisting of sodium chloride, potassium chloride, magnesium chloride, calcium chloride, ammonium sulfate, and mixture thereof.

The sugar is selected from the group consisting of glucose, sucrose, trehalose, mannose, fructose, arabinose, galactose, xylose, ribose, dextran, cycloamylose, cyclodextrin, and mixture thereof.

The alcohol compound is selected from the group consisting of propanol, methanol, ethanol, butanol, phenol, glycerol, ethylene glycol, propylene glycol, polyethylene glycol, polypropylene glycol, copolymer of polyethylene glycol and polypropylene glycol, and mixture thereof.

The surfactant is selected from the group consisting of 2-[4-(2,4,4-trimethylpentan-2-yl)phenoxy]ethanol), 2-[2-[4-(2,4,4-trimethylpentan-2-yl)phenoxy]ethoxy]ethanol, sodium dodecyl sulfate, 3-[(3-cholamidopropyl) dimethylammonio]-1-propanesulfonate (CHAPS), cetyltrimethylammonium bromide (CTAB), N-cetyltrimethylammonium chloride, alkylpoly ethylene glycol ether, octaethylene glycol monododecyl ether, polyoxyethylene lauryl ether, polyethylene glycol hexadecyl ether, octyl β-D-glucopyranoside, dodecyl maltoside, polyvinyl alcohol, nonylphenyl-polyethylene glycol, octanoyl-N-methylglucamide, nonanoyl-N-methyiglucamine, decanoyl-N-methylglucamide, N,N-Bis(3-D-gluconamidopropyl) deoxycholamide, chenodeoxycholic acid, cholic acid, deoxycholic acid, N-lauroylsarcosin, lauric acid, and mixture thereof.

The aggregate suppressor is selected from the group consisting of proline, glycine, arginine, lysine, alanine, leucine, isoleucine, valine, phenylalanine, tyrosine, tryptophan, asparagine, glutamic acid, glutamine, aspartic acid, serine, threonine, cysteine, methionine, histidine, sodium chloride, potassium chloride, magnesium chloride, calcium chloride, ammonium sulfate, glucose, sucrose, trehalose, mannose, fructose, arabinose, galactose, xylose, ribose, dextran, cycloamylose, cyclodextrin, propanol, methanol, ethanol, butanol, phenol, glycerol, ethylene glycol, propylene glycol, polyethylene glycol, polypropylene glycol and copolymer of polyethylene glycol and polypropylene glycol, 2-[4-(2,4,4-trimethylpentan-2-yl)phenoxy]ethanol), 2-[2-[4-(2,4,4-trimethylpentan-2-yl)phenoxy]ethoxy]ethanol, sodium dodecyl sulfate, 3-[(3-cholamidopropyl)dimethylammonio]-1-propanesulfonate, cetyltrimethylammonium bromide, N-cetyltrimethylammonium chloride, alkylpoly ethylene glycol ether, octaethylene glycol monododecyl ether, polyoxyethylene lauryl ether, polyethylene glycol hexadecyl ether, octyl β-D-glucopyranoside, dodecyl maltoside, polyvinyl alcohol, nonylphenyl-polyethylene glycol, octanoyl-N-methylglucamide, nonanoyl-N-methylglucamine, decanoyl-N-methylglucamide, N,N-Bis(3-D-gluconamidopropyl) deoxycholamide, chenodeoxycholic acid, cholic acid, deoxycholic acid, N-lauroylsarcosin, lauric acid, 2-(cyclohexylamino)ethanesulfonic acid, 3-(1-pyridinio)-1-propanesulfonate, pyridine-3-sulfonic acid, nicotinic acid, 3-[(3-cholamidopropyl) dimethylammonio]-1-propanesulfonate (CHAPS), 3-(3-cholamidopropyl) dimethylammonio-2-hydroxy-1-propane sulfonate, and mixture thereof. The preferred aggregate suppressor is arginine.

Preferred proteins for regeneration in this invention are from transforming growth factor beta superfamily (TGF-β superfamily) which is selected from the group consisting of bone morphogenetic protein, transforming growth factor beta (TGF-β), activins, growth differentiation factor (GDF), derivatives of bone morphogenetic protein, derivatives of TGF-β, derivatives of activins, derivatives of GDF, fusions of bone morphogenetic protein, fusions of TGF-β, fusions of activins, fusions of GDF, genetic modification of bone morphogenetic protein, genetic modification of TGF-β, genetic modification of activins, genetic modification of GDF, and mixture thereof.

Use of polysorbate as a secondary additive in the renaturation buffer solution can synergistically enhance performance of primary additives, here including a mixture of reducing and oxidizing thiol compounds, folding enhancers and aggregate suppressors. The synergistic effect is to quantitatively and qualitatively promote dimerization, e.g., having a greater rate and possessing a more bioactive conformation, with no increase in redox system concentration and no impact on purification ability of the proteins. This benefits in reducing production cost and cycle time. Application of the said renaturation buffer solution with the method of the present invention can increase after purification yield of each production cycle by 7 folds and biological activity by about 2 time, as compared to that produced using the prior art method, reported by von Einem S. and coworkers (von Einem S, et al., Protein Expr Purif 2010; 73: 65-69).

The detailed description and examples illustrate various embodiments and explain the principles of the compositions of renaturation buffer solution and the method for protein renaturation of the present invention, without being meant to be limitative.

The method for renaturation of dimeric proteins of the present invention comprises the following steps.

### Step 1 Dissolving a Denatured Protein in a Solubilization Buffer Solution

Denatured protein is first dissolved in a solubilization buffer solution to a concentration of 1-20 mg/mL at a temperature of 0-30° C. for 1-24 hours or until the protein folding is completely destroyed. The used period of time depends on types of target protein and solubilization buffer solution. After adjusting pH to 3-4, the obtained protein solution is dialyzed to remove the remained reducing agent and then concentrated using ultrafiltration techniques, such as crossflow filtration, to a final concentration of 20-60 mg/mL. The solution is kept at 4° C. until use in the next step.

The preferred condition for denatured protein dissolution is 25° C. for 2 hours.

The composition of the solubilization buffer solution comprises:
a) 20-250 mM buffer at pH range of 2-12,
b) 5-200 mM denaturant,
c) 5-200 mM reducing agent for disulfide bond formation.

The composition of the solubilization buffer solution further comprises chelating agent, aggregate suppressor, and mixture thereof. The composition of the solubilization buffer solution can be modified upon type of target proteins and user's convenience.

A buffer is selected from Good's buffers.

A preferred Good's buffer is selected from the group consisting of Tris buffer, HEPES buffer, phosphate buffer, MES buffer, tricine buffer, and mixture thereof.

A denaturant is selected from the group consisting of guanidine hydrochloride, urea, thiourea, 2-mercaptoethanol, dithiothreitol, tris(2-carboxyethyl)phosphine, and mixture thereof.

A reducing agent for disulfide bond formation is selected from the group consisting of dithiothreitol, dithioerythritol, 2-mercaptoethanol, tris(2-carboxyethyl)phosphine, reduced glutathione, and mixture thereof.

A chelating agent is selected from the group consisting of ethylenediaminetetraacetic acid (EDTA), pentetic acid or diethylenetriaminepentaacetic acid (DTPA), N-(2-hydroxyethyl)ethylenediaminetriacetic acid (HEDTA), egtazic acid or ethylenebis(oxyethylenenitrilo)tetraacetic acid (EGTA), nitrilotriacetic acid (NTA), dimercaptosuccinic acid (DMSA), 2,3-dimercapto-1-propanol (DMP), penicillamine, and mixture thereof.

An aggregate suppressor is selected from the group consisting of proline, glycine, arginine, lysine, alanine, leucine, isoleucine, valine, phenylalanine, tyrosine, tryptophan, asparagine, glutamic acid, glutamine, aspartic acid, serine, threonine, cysteine, methionine, histidine, sodium chloride, potassium chloride, magnesium chloride, calcium chloride, ammonium sulfate, glucose, sucrose, trehalose, mannose, fructose, arabinose, galactose, xylose, ribose, dextran, cycloamylose, cyclodextrin, propanol, methanol, ethanol, butanol, phenol, glycerol, ethylene glycol, propylene glycol, polyethylene glycol, polypropylene glycol, copolymer of polyethylene glycol and polypropylene glycol, 2-[4-(2,4,4-trimethylpentan-2-yl)phenoxy]ethanol, 2-[2-[4-(2,4,4-trimethylpentan-2-yl)phenoxy]ethoxy]ethanol, sodium dodecyl sulfate, 3-[(3-cholamidopropyl) dimethylammonio]-1-propanesulfonate, cetyltrimethylammonium bromide, N-cetyltrimethylammonium chloride, alkylpoly ethylene glycol ether, octaethylene glycol monododecyl ether, polyoxyethylene lauryl ether, polyethylene glycol hexadecyl ether, octyl β-D-glucopyranoside, dodecyl maltoside, polyvinyl alcohol, nonylphenyl-polyethylene glycol, octanoyl-N-methylglucamide, nonanoyl-N-methylglucamine, decanoyl-N-methylglucamide, N,N-Bis(3-D-gluconamidopropyl) deoxycholamide, chenodeoxycholic acid, cholic acid, deoxycholic acid, N-lauroylsarcosin, lauric acid, 2-(cyclohexylamino)ethanesulfonic acid, 3-(1-pyridinio)-1-propanesulfonate, pyridine-3-sulfonic acid, nicotinic acid, 3-[(3-cholamidopropyl)-dimethylammonio]-1-propane sulfonate, 3-(3-cholamidopropyl) dimethylammonio-2-hydroxy-1-propane sulfonate, and mixture thereof.

### Step 2 Diluting the Denatured Protein Solution Using Renaturation Buffer Solution Containing Polysorbate Compound

The denatured protein solution from Step 1 is diluted with a renaturation buffer solution containing polysorbate compound to a final concentration of 0.05-0.5 mg/mL in a presence of 0.05-1 mg/mL reducing thiol at a temperature of 0-25° C. for 1-24 hours. This is to optimally allow the target protein to fold and adopt correct secondary and tertiary conformations. This method step is referred to as “Refolding”.

The preferred condition for denatured protein dilution is at a temperature of 4° C. for 24 hours.

Compositions of the renaturation buffer solution comprise 20-250 mM buffer at pH range of 6-10, a mixture of reducing and oxidizing thiol compounds at a concentration of 0.05-5 mM and preferred molar ratios of reduced and oxidized thiol of 20:1 to 1:20, 0.1-5 M primary additives and 0.005-0.2% V polysorbate compound.

The buffer is selected from Good's buffers.

The preferred Good's buffer is selected from the group consisting of Tris buffer, HEPES buffer, phosphate buffer, MES buffer, tricine buffer, and mixture thereof. The preferred buffer is Tris buffer. The preferred concentration of buffer is 100 mM and the preferred pH is 8-9.

The oxido shuffling system, or mixture of reducing and oxidizing thiol compounds, is selected from the group consisting of a reduced glutathione (GSH)/oxidized glutathione (GSSG) mixture, a cysteine/cystine mixture, a cysteamine/cystamine mixture, a 2-mercaptoethanol/2-hydroxyethyl disulfide mixture, and mixture thereof. The preferred mixture of reducing and oxidizing thiol is a reduced glutathione and oxidized glutathione mixture or a cysteine and cystine mixture. The preferred concentration of mixture of reducing and oxidizing thiol compounds is 0.05-1 mM and a molar ratio of reduced and oxidized thiol of 10:1 to 1:10.

The primary additive is selected from the group consisting of arginine, glutamate, 2-(cyclohexylamino)ethanesulfonic acid (CHES), 3-(1-pyridinio)-1-propanesulfonate pyridine-3-sulfonic acid, nicotinic acid, 3-[(3-cholamidopropyl)-dimethylammonio]-1-propane sulfonate (CHAPS), 3-(3-cholamidopropyl) dimethylammonio-2-hydroxy-1-propane sulfonate (CHAPSO), cholic acid, deoxycholic acid, taurocholic acid, taurodeoxycholic acid, digitonin, and mixture thereof. The preferred primary additive is arginine. The preferred concentration of primary additive is 0.25-3 M.

The polysorbate compound is selected from the group consisting of polysorbate 20, polysorbate 40, polysorbate 60, polysorbate 80, polysorbate 85, and mixture thereof. The preferred polysorbate compound is polysorbate 80 at a concentration of 0.05% V/V.

The chelating agent is selected from the group consisting of ethylenediaminetetraacetic acid (EDTA), pentetic acid or diethylenetriaminepentaacetic acid (DTPA), N-(2-hydroxyethyl)ethylenediaminetriacetic acid (HEDTA), egtazic acid or ethylenebis(oxyethylenenitrilo)tetraacetic acid (EGTA), nitrilotriacetic acid (NTA), dimercaptosuccinic acid (DMSA), 2,3-dimercapto-1-propanol (DMP), penicillamine, and mixture thereof. The preferred chelating agent is ethylenediaminetetraacetic acid at a concentration of 5 mM.

### Step 3 Concentrating the Protein Solution to a Concentration Approaching Saturation

Formation of disulfide bonded dimer can be accelerated by concentrating the previously diluted protein solution with the renaturation buffer to a concentration approaching saturation at a temperature of 0-25° C. using an ultrafiltration technique that is selected from crossflow filtration technique, centrifugal filter device, and mixture thereof. The obtained concentrated protein solution is allowed to incubate at 0-25° C. for 1-48 hours. This method step is referred to as “Dimerization”.

The preferred condition for concentrating the protein solution is at a temperature of 4° C. for 24 hours. Further incubation of another 24 hours will accelerate inter-chain disulfide bond formation, resulting in dimer of more than 85 percent of the total target protein.

Since the amount of polysorbate, which acts as a dimerization enhancer in the renaturation buffer solution according to this invention, is not sufficiently high to significantly change surface binding property of the protein and affect purification performance by chromatography techniques. Moreover, biocompatible polysorbates are surfactants that can stabilize proteins against surface adsorption and are commonly used as a stabilizer for proteins in food and pharmaceutical products. Therefore, there is no need to employ additional technical steps to remove polysorbate from the target protein solution prior to dimer separation from monomer and other protein contaminants. This will reduce steps and time required to renaturate the protein.

Recombinant BMP-2 was used as a protein model for studying renaturation of dimeric biologically active proteins using the renaturation buffer solution containing compositions of the invention. The study results are as follows:

### 1. Screening Experiment of the Composition of Renaturation Buffer Solution Effectively Suitable for the Recombinant BMP-2 Protein

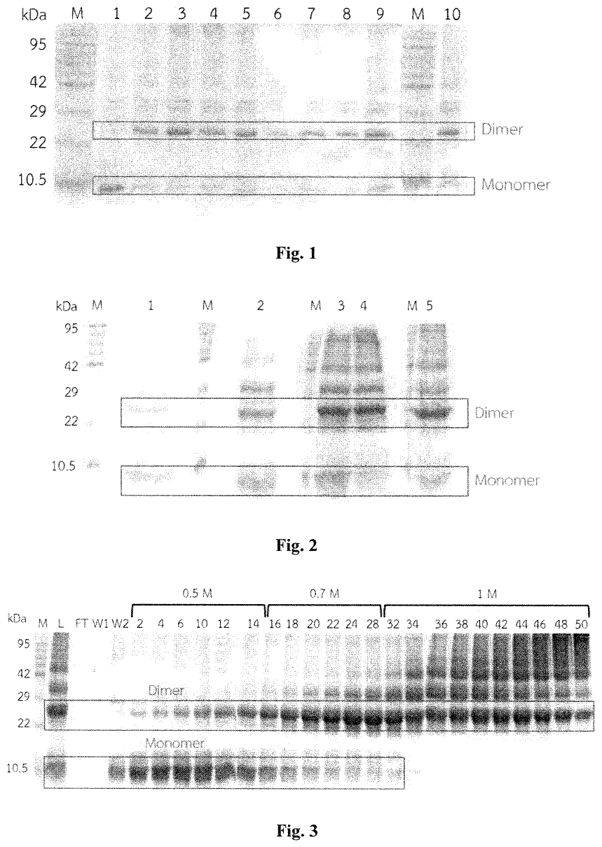

In this invention, polysorbate compounds are used as secondary additives to synergistically enhance the efficiency of primary additives, which here refers to arginine. This screen test aimed to identify a concentration range and types of polysorbate compounds suitable for the renaturation buffer used for renaturation of the recombinant BMP-2. The renaturation process was as follows:
a) Recombinant BMP-2 inclusion body was dissolved in 100 mM Tris buffer solution (pH 8.5) containing 6 M guanidine hydrochloride, 1 mM ethylenediaminetetraacetic acid (EDTA), 100 mM dithiothreitol at room temperature for 2 hours to obtain a recombinant BMP-2 solution with a concentration of 10 mg/mL. The pH of the obtained protein solution was adjusted to 3-4 with 25% W/V hydrochloric acid solution to prevent disulfide formation. After centrifuging at 14,000 rpm and 4° C. for 20 minutes, the supernatant was dialyzed against 4-5 changes of a 20 folded excess of 6 M urea solution at 4° C. and then concentrated to a final concentration of 20-30 mg/mL.
b) The protein solution obtained from Step a) was diluted using a renaturation buffer having a following composition: 100 mM Tris buffer solution (pH 8.3) containing 0.1 mM oxidized glutathione, 0.1 mM reduced glutathione, 5 mM ethylenediaminetetraacetic acid (EDTA), and arginine and polysorbate at concentrations listed in Table 1. The final concentration of the protein solution was 200 μg/mL. The obtained protein solution was stirred at 4° C. for 24 hours and centrifuged at 14,000 rpm and 4° C. for 20 minutes to remove the insoluble part.
c) Dimerization was initiated by increasing the concentration of the protein solution from Step b) to a final concentration of 500 pig/mL using crossflow filtration technique. After stirring at 4° C. for 0, 24 and 48 hours, 20 μL of protein solutions were sampling and precipitated using trichloroacetic acid. The obtained protein was characterized using sodium dodecyl sulfate polyacrylamide gel electrophoresis (SDS-PAGE) and quantitatively analyzed using ImageJ software. Renaturation yields (%), expressed as percentage of dimerized targeted protein compared to total targeted protein in both monomer and dimer forms, calculated and shown in FIG. 1 and Table 1.

TABLE 1
Percentages of renaturation yields of recombinant BMP-2 under different conditions.

| | | | | Percentages of renaturation | | |
|---|---|---|---|---|---|---|
| | Additives | | | yields after dimerization | | |
| | Arginine | Polysorbate 20 | Polysorbate 80 | 0 | 24 | 48 |
| Experiment | (M) | (%) | (%) | hour | hours | hours |
| 1 | 0 | 0 | 0.05 | 0 | 0 | 0 |
| 2 | 1 | 0 | 0 | 0.56 | 0.54 | 0.68 |
| 3 | 1 | 0.010 | 0 | 0.65 | 0.68 | 0.73 |
| 4 | 1 | 0.050 | 0 | 0.63 | 0.69 | 0.78 |
| 5 | 1 | 0.100 | 0 | 0.65 | 0.69 | 0.70 |
| 6 | 1 | 0 | 0.010 | 0.62 | 0.75 | 0.73 |
| 7 | 1 | 0 | 0.025 | 0.60 | 0.82 | 0.82 |
| 8 | 1 | 0 | 0.050 | 0.59 | 0.82 | 0.83 |
| 9 | 1 | 0 | 0.075 | 0.65 | 0.83 | 0.91 |
| 10 | 1 | 0 | 0.100 | 0.60 | 0.81 | 0.86 |

Note
Oligomer larger than dimer and aggregates of targeted proteins cannot be quantitatively determined and, therefore, cannot be included in the calculation.

The result of screening experiment shows that arginine acted as a primary additive and polysorbate acted as a secondary additive. Addition of polysorbate to the renaturation buffer could effectively stimulate dimerization of recombinant BMP-2 protein and a concentration of 0.05% and higher was required to obtain renaturation yield greater than 80% after 24 hours dimerization. A renaturation yield greater than 90% was received after 48 hours dimerization in a buffer with a concentration of polysorbate 80 of 0.075%©. Additionally, polysorbate 20 showed comparable promotion of dimerization and this implied that polysorbate compounds can be used to synergistically enhance efficiency of primary additives in the renaturation buffer solution.

### 2. Renaturation Efficiency of Recombinant BMP-2 Using Renaturation Buffer Composition and Method of the Present Invention

The following examples, illustrating the protein renaturation using the method of the invention, used recombinant BMP-2 as a protein model. A renaturation buffer solution containing polysorbate 80 at concentrations of 0.05% and 0.075% was used to renaturate inclusion body produced from a 2 L culture. The method is as follows:

### 1. Dissolving the Denatured Protein

BMP-2 inclusion body was dissolved in 100 mM Tris buffer solution (pH 8.5) containing 6 M guanidine hydrochloride, 1 mM ethylenediaminetetraacetic acid (EDTA), 100 mM dithiothreitol (DTT) at room temperature for 2 hours to a concentration of 10 mg/mL. The protein solution pH was adjusted to 3-4 with 25% WN hydrochloric acid to prevent disulfide bond formation and then centrifuged at a speed of 14,000 rpm and a temperature of 4° C. for 20 minutes. The obtained supernatant was dialyzed against 20-fold volume of 6 M guanidine hydrochloride solution and then concentrated to a final concentration of 20-30 mg/mL.

### 2. Diluting the Denatured Target Protein Solution (Refolding)

The protein solution was diluted to different final concentrations as shown in Table 1 using 100 mM Tris buffer solution (pH 8.3) containing 5 mM ethylenediaminetetraacetic acid (EDTA), 1 M arginine, 0.1 mM oxidized glutathione or cystine, 0.1 mM reduced glutathione or cysteine and polysorbate 80 at a concentration of 0.050% and 0.075%. The obtained protein solution was stirred at 4° C. for 24 hours and then centrifuged at a speed of 14,000 rpm and a temperature of 4° C. for 20 minutes to discard the insoluble part.

### 3. Concentrating the Protein Solution to a Concentration Approaching Saturation (Dimerization

The target protein solution was concentrated to a concentration of 1 mg/mL using crossflow filtration technique with molecular weight cut off (MWCO) of 10,000 Daltons and then stirred at 4° C. for 48 hours. The obtained protein was characterized by sodium dodecyl sulfate-polyacrylamide gel electrophoresis technique and quantitatively analyzed using ImageJ software. Renaturation yields (%), expressed as percentage of dimerized targeted protein compared to total targeted protein in both monomer and dimer forms, are calculated and shown in Table 2. Note that oligomer larger than dimer and aggregates of targeted proteins cannot be quantitatively determined and, therefore, cannot be included in the calculation. FIG. 2 showed effect of polysorbate 80 addition in the renaturation buffer solution of the present invention in the “Dimerization” step of the method of this invention using SDS-PAGE under nonreducing conditions and without heating. A single band of BMP-2 dimer may indicate that most of the dimeric protein possesses the similar conformation.

### 4. Purification of Recombinant BMP-2 Renaturated Using the Method of the Present Invention

A chromatographic separation of the monomeric and dimeric forms of the recombinant BMP2 was carried out using heparin affinity chromatography technique. The renaturated protein solution was dialyzed against 100 mM Tris buffer solution (pH 6) containing 5 mM ethylenediaminetetraacetic acid (EDTA) and 6 M urea prior to passing through the heparin sepharose column (FIG. 3). The result shows that BMP-2 monomer was eluted from the column with a salt solution faster than its dimer.

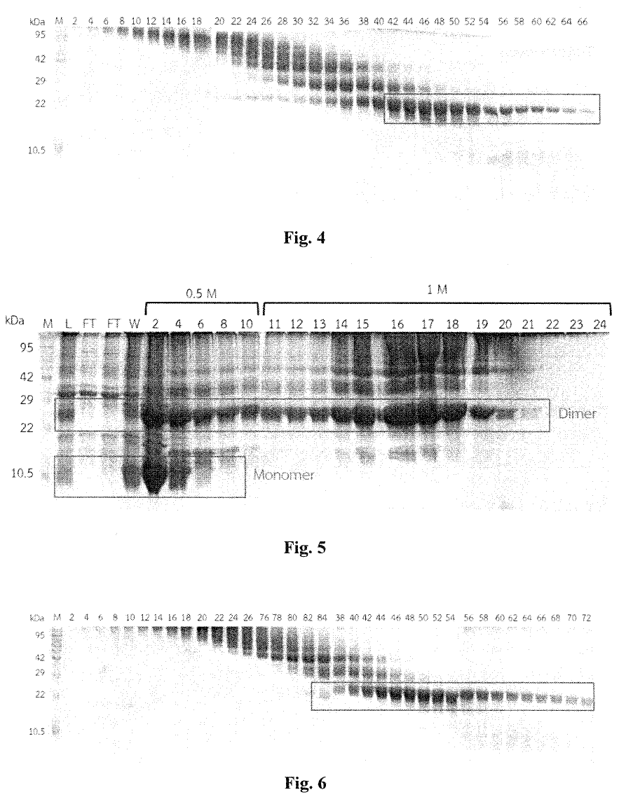

The 2 mL eluted fractions containing the recombinant BMP-2 dimer (fraction 41-66) were pooled and further purified using Superdex 200 size-exclusion chromatography (Table 2 and FIG. 4). The result shows that the control experiment (Experiment Number 1 in Table 2), using renaturation buffer solution without the secondary polysorbate additive, exhibited very low renaturation yield after stirring in the “Dimerization” step for 48 hours. This finding is consistent with a prior art (Vallejo L F, et al., Biotechnol Bioeng. 2004; 85: 601-609) and another prior art reported that it took 14 days in order to get a renaturation yield of 50% (Hillger F, et al., J Biol Chem. 2005; 280: 14974-14980). Combination of the method of the present invention with the addition of polysorbate 80 at a concentration of 0.05% resulted in a renaturation yield of 80% after 24 hours in the “Refolding” step followed by concentrating the protein solution to a concentration approaching saturation and stirring for another 24 hours in the “Dimerization” step.

Utilization of polysorbate 80 as a secondary additive can synergistically enhance performance of arginine in renaturation as seen in obtaining renaturation yield of 50%-80% after 48 hours stirring in the “Dimerization” step (Table 2 Experiment number 2-5). The renaturation buffer solution of this present invention with polysorbate at a concentration of 0.050% showed greater dimerization efficiency than that at a concentration of 0.075%. Additionally, the second step of this invention, dilution of denatured target protein solution, was found to be important for correct folding and/or suppression of protein aggregation. As found that using protein solution with a high concentration of 1 mg/mL in the “Refolding” step, equal to that used in the “Dimerization” step, resulted in unwanted precipitation of all of the target protein in the “Refolding”. Total yield, listed in Table 2, is the amount of protein collected after purification with heparin affinity and size exclusion chromatography. The absence of polysorbate in the renaturation buffer of this invention leaded to a very low total yield after the affinity chromatographic purification and the further purification by size exclusion chromatography was not practical. This is due to significant precipitation during renaturation, dialysis for media exchange and pH adjustment prior to passing through heparin affinity column. Therefore, the method in the present invention requires a renaturation buffer solution containing polysorbate.

TABLE 2
Percentages of renaturation yields and total yields after purification of recombinant
BMP-2 prepared using different compositions of renaturation buffers

| | | Protein | Percentages of | | | | |
|---|---|---|---|---|---|---|---|
| | Polysorbate | concentration | renaturation yields after | | | Total yields after | |
| | 80 | in | “Dimerization” | | | purification (mg) | |
| | concentration | “Refolding” | 0 | 24 | 48 | Heparin | Superdex |
| Experiment | (%) | (mg/mL) | hour | hours | hours | sepharose | 200 |
| | Method of the present invention | | | | | | |
| 1 | 0 | 0.2 | 33 | n/d | 38 | 2.27 | n/p |
| 2 | 0.050 | 0.2 | 71 | 81 | 83 | 48.75 | 15.65 |
| 3 | 0.075 | 0.2 | 35 | n/d | 50 | 6.18 | 2.93 |
| 4 | 0.075 | 0.5 | 37 | n/d | 51 | 4.83 | n/p |
| 5 | 0.075 | 1.0 | 0 | n/d | 0 | n/p | n/p |
| | Prior art method (von Einem S, et al., Protein Expr Purif. 2010; 73: 65-69) | | | | | | |
| 6 | 0 | 0.2 | n/d | 45 | 61 | 11.78 | 2.17 |

n/d = not determined
n/p = not performed

### 3. Recombinant BMP-2 Renaturation Using Prior Art Method

Recombinant BMP-2 inclusion body was renaturated using the method and the renaturation buffer solution of the prior art reported by Von Einem and coworkers (von Einem S, et al., Protein Expr Purif. 2010; 73: 65-69) and the results are shown in Table 2 (Experiment number 6). Renaturation yield of recombinant BMP-2 protein refolded by the prior art method is less than the method according to this invention. Elution pattern of the heparin affinity chromatography (FIG. 5) clearly exhibited the coexistence of two different dimeric BMP-2 refolded by the prior art method. A portion of BMP-2 dimer (Column fraction 2-10 in FIG. 5) was co-eluted from the column along with its monomer and tested to have no in vitro biological activity. BMP-2 dimer with the bioactive conformation was eluted latter (Column fraction 11-21 in FIG. 5) and further purified using size exclusion chromatography (FIG. 6). In contrast, the BMP-2 dimer obtained from the renaturation method of the present invention are continuously eluted from the heparin affinity column, with no increase or decrease in the protein band intensity that clearly indicated elution of different dimer conformation. Most of the BMP-2 dimer was eluted in the column fraction 16-50 and tested to be bioactive (FIG. 3). The total yield after purified with heparin chromatography of the recombinant BMP-2 refolded using the method of this invention increased approximately 4 folds, as compared to that refolded by the prior art method (Table 2).

Moreover, the protein obtained from the present invention formed undesired oligomer larger than dimer in a less degree, as compared to that from the prior art method, leading to a greater total yield after purification using Superdex 200 size-exclusion chromatography at approximately 7.21 folds.

### 4. Comparison of Biological Activity of Recombinant BMP-2 Renatured Using the Method of the Present Invention and the Prior Art Method

Biological activity of the recombinant BMP-2 was evaluated through ability to promote osteoblast differentiation of C2Cl2 mouse myoblast cell line. To examine protein-induces alkaline phosphatase activity, the myoblast C2Cl2 cells was incubated in a 96-well cell culture plate at a concentration of 3×10⁵ cells/mL in Dulbecco's Modified Eagle Medium (DMEM) supplemented with 10% fetal bovine serum for 24 hours at 37° C. under 5% CO₂ atmosphere for 24 hours. After cell washing, cells in each well were treated with 0.1 mL DMEM culture media containing 2% horse serum and tested recombinant protein at different concentrations (3, 25 and 100 μg/mL). Alkaline phosphatase was then quantified after BMP-2 treatment at 37° C. under 5% CO₂ atmosphere for 3 days.

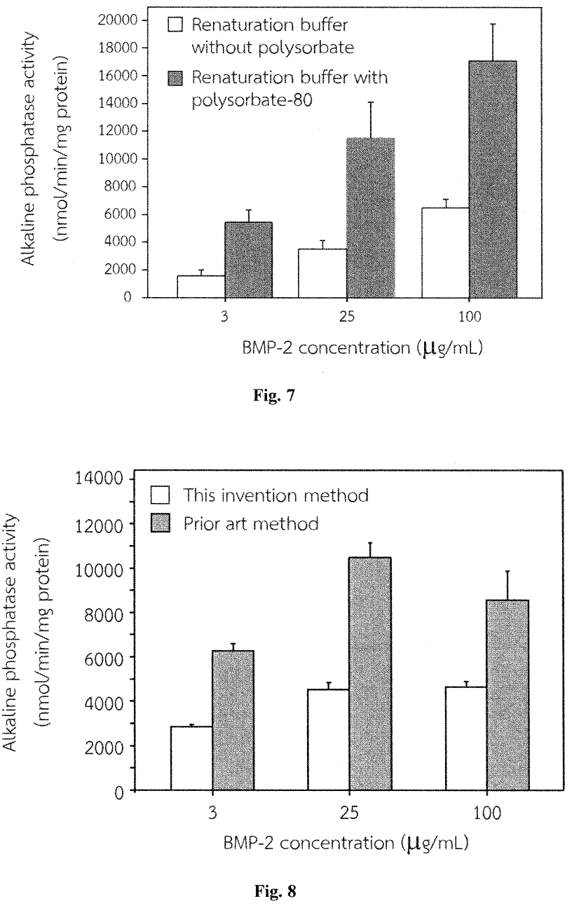

The results in FIG. 7 and Table 2 indicate that the use of polysorbate 80 as a secondary additive synergistically enhance arginine effectiveness in the renaturation buffers. Addition of polysorbate 80 at a concentration of 0.05% resulted in more than doubly increased dimerization, as compared to that of a polysorbate-free renaturation buffer, and renaturation yield of more than 80% was obtained after 24 hour dimerization. This appeared to be more effective than renaturation buffer solutions for dimeric protein previously reported in the prior arts to be in the range of 15% — 63% (Gieseler G M, et al., Biotechnol Rep (Amst). 2018; 18: e00249; Gieseler G M, et al., Appl Microbiol Biotechnol. 2017; 101: 123-130; Vallejo L F, et al., Biotechnol Bioeng. 2004; 85: 601-609; Vallejo L F, et al., J Biotechnol. 2002; 94: 185-194; Hillger F, et al., J Biol Chem. 2005; 280: 14974-14980; Honda J, et al., J Biosci Bioeng. 2000; 89: 582-589; von Einem S, et al., Protein Expr Purif. 2010; 73: 65-69). A renaturation buffer with a low concentration of oxidizing agents, reported by von Einem and coworkers, could provide a renaturation yield of about 40%-50% for BMP-2 renaturation using a similar process to the one according to this invention (von Einem S, et al., Protein Expr Purif. 2010; 73: 65-69). To promote interchain disulphide bond formation, von Einem and his team added another renaturation step using a buffer having a high concentration of oxidizing agents and the renaturation yield increased to about 70%-80%. However, about half of the renaturated BMP-2 was misfolding and/or incorrect dimerization and the reported percentages of renaturation yield were evaluated using combining amounts of both correct and incorrect folded protein.

Protein precipitation problem leaded to less renaturation and total yields (i.e., the amount of protein collected after purification) obtained from the renaturation buffer of this invention without and with polysorbate 80 at a concentration of 0.075% V, as compared to that at a concentration of 0.050% V. Such precipitation became more common with increasing protein concentration in the dimerization step of this invention. Without adding polysorbate to the renaturation buffer, a relatively large amount of precipitate was found during renaturation and exchange to polysorbate-free media prior to passing through heparin affinity column. The total yield obtained after heparin affinity column was too low for further purification by size exclusion chromatography.

Polysorbate 80 addition at both tested concentrations (0.050% V and 0.075% V) to the renaturation buffer was found to effectively suppress aggregation during the high-concentration dimerization and also promote disulfide formation. However, extreme protein aggregation unexpectedly occurred only in the case of renaturation buffer with 0.075% V polysorbate 80 during exchange to polysorbate-free media prior to passing through heparin affinity column. Decreasing the polysorbate 80 concentration to 0.050% V could prevent such precipitation and, therefore, a suitable polysorbate concentration in the renaturation buffer could increase the total yield after heparin affinity chromatography up to 21.5 folds.

Biological activities of renatured recombinant BMP-2 are shown in FIG. 7 and FIG. 8. The recombinant BMP-2 renatured according to this invention stimulate osteogenic differentiation of C2Cl2 myoblast cells. Alkaline phosphatase activity increased in a concentration-dependent manner from 3 to 25 μg/mL and started to plateau at a concentration of 100 μg/mL. Addition of polysorbate in the renaturation buffer of this invention was found to enhance osteoblast differentiation (biological activity) of the recombinant BMP-2 at approximately 2.5-3 folds over the entire range of tested concentrations (3-100 μg/mL). Moreover, the recombinant BMP-2 renaturated according to the invention was found to have approximately 2-fold greater osteoblast differentiation (biological activity) than BMP-2 produced by the prior art method (p<0.05) over the entire range of tested concentrations.

### BEST MODE FOR CARRYING OUT THE INVENTION

As mentioned in the topic of Disclosure of Invention.

1-21. (canceled)

22. A method of renaturation dimeric proteins comprising:
a) dissolving a denatured protein in a solubilization buffer solution to a final concentration of 20-60 mg/mL;
b) diluting the denatured protein solution from a) using a renaturation buffer solution containing polysorbate compound at a temperature ranging from 0-25° C. for 1-24 hours to a final concentration ranging from 0.05-0.75 mg/mL, said renaturation buffer solution comprising 20-250 mM buffer at pH range of 6-10, a 20:1 to 1:20 mixture of reducing and oxidizing thiol at a concentration of 0.01-5 mM, 0.1-5 M primary additive and 0.02-0.2% V polysorbate compound; and
c) concentrating the protein solution obtained from b) to a concentration approaching saturation and stirring at a temperature ranging from 0-25° C. for 1-48 hours.

23. The method of claim 22, further comprising the step of purifying the protein using heparin affinity and size exclusion chromatography.

24. The method of claim 22, wherein said dissolving is carried out by dissolving the denatured protein in a solubilization buffer to a final protein concentration of 1-20 mg/mL at a temperature ranging from 0-30° C. for 1-48 hours, the method further comprising:
adjusting the pH of the protein solution to a range of 3-4;
removing the reducing thiol using dialysis; and
increasing the protein concentration to a final concentration of 20-60 mg/mL.

25. The method of claim 24, wherein dissolving is performed at a temperature of 25° C. for 2 hours.

26. The method of claim 22, wherein the solubilization buffer solution comprises: a) 20-250 mM buffer at pH range of 2-12; b) 5-200 mM denaturant; and c) 5-200 mM reducing agent for disulfide bond formation.

27. The method of claim 26, wherein the solubilization buffer solution further comprises chelating agent, aggregate suppressor, and mixture thereof.

28. The method of claim 22, wherein diluting is performed at a temperature of 4° C. for 24 hours.

29. The method of claim 22, wherein the renaturation buffer solution further comprises chelating agents at a concentration of 1-10 mM.

30. The method of claim 29, wherein the renaturation buffer solution comprises:
a) 100 mM buffer at pH range of 8-9;
b) a mixture of reducing and oxidizing thiol at a concentration of 0.05-1 mM and a molar ratio of reduced and oxidized thiol of 10:1 to 1:10;
c) 0.25-3 M primary additive; and
d) 0.05% V polysorbate compound; e) 5 mM chelating agent.

31. The method of claim 22, wherein the buffer in the solubilization buffer solution and the renaturation buffer solution is a Good's buffer.

32. The method of claim 31, wherein the Good's buffer is selected from the group consisting of Tris buffer, HEPES buffer, phosphate buffer, MES buffer, tricine buffer, and mixture thereof.

33. The method of claim 26, wherein the denaturant is selected from the group consisting of guanidine hydrochloride, urea, thiourea, 2-mercaptoethanol, dithiothreitol, tris(2-carboxyethyl)phosphine, and mixture thereof.

34. The method of claim 26, wherein the reducing agent is selected from the group consisting of dithiothreitol, dithioerythritol, 2-mercaptoethanol, tris(2-carboxyethyl)phosphine, reduced glutathione, and mixture thereof.

35. The method of claim 27, wherein the aggregate suppressor is selected from the group consisting of proline, glycine, arginine, lysine, alanine, leucine, isoleucine, valine, phenylalanine, tyrosine, tryptophan, asparagine, glutamic acid, glutamine, aspartic acid, serine, threonine, cysteine, methionine, histidine, sodium chloride, potassium chloride, magnesium chloride, calcium chloride, ammonium sulfate, glucose, sucrose, trehalose, mannose, fructose, arabinose, galactose, xylose, ribose, dextran, cycloamylose, cyclodextrin, propanol, methanol, ethanol, butanol, phenol, glycerol, ethylene glycol, propylene glycol, polyethylene glycol, polypropylene glycol, copolymer of polyethylene glycol and polypropylene glycol, 2-[4-(2,4,4-trimethylpentan-2-yl)phenoxy]ethanol, 2-[2-[4-(2,4,4-trimethylpentan-2-yl)phenoxy]ethoxy]ethanol, sodium dodecyl sulfate, 3-[(3-cholamidopropyl) dimethylammonio]-1-propanesulfonate, cetyltrimethylammonium bromide, N-cetyltrimethylammonium chloride, alkylpoly ethylene glycol ether, octaethylene glycol monododecyl ether, polyoxyethylene lauryl ether, polyethylene glycol hexadecyl ether, octyl b-D-glucopyranoside, dodecyl maltoside, polyvinyl alcohol, nonylphenyl-polyethylene glycol, octanoyl-N-methylglucamide, nonanoyl-N-methylglucamine, decanoyl-N-methylglucamide, N,N-Bis(3-D-gluconamidopropyl) deoxycholamide, chenodeoxycholic acid, cholic acid, deoxycholic acid, N-lauroylsarcosin, lauric acid, 2-(cyclohexylamino)ethanesulfonic acid, 3-(1-pyridinio)-1-propanesulfonate, pyridine-3-sulfonic acid, nicotinic acid, 3-[(3-cholamidopropyl)-dimethylammonio]-1-propane, 3-(3-cholamidopropyl) dimethylammonio-2-hydroxy-1-propane sulfonate, and mixture thereof.

36. The method of claim 22, wherein the mixture of reducing and oxidizing thiol is selected from the group consisting of a reduced glutathione and oxidized glutathione mixture, a cysteine and cystine mixture, a cysteamine and cystamine mixture, a 2-mercaptoethanol and 2-hydroxyethyl disulfide mixture, and mixture thereof.

37. The method of claim 22, wherein primary additive is selected from the group consisting of arginine, glutamate, 2-(cyclohexylamino)ethanesulfonic acid, 3-(1-pyridinio)-1-propanesulfonate, pyridine-3-sulfonic acid, nicotinic acid, 3-[(3-cholamidopropyl)-dimethylammonio]-1-propane sulfonate, 3-(3-cholamidopropyl)-dimethylammonio-2-hydroxy-1-propane sulfonate, cholic acid, deoxycholic acid, taurocholic acid, taurodeoxycholic acid, digitonin, and mixture thereof.

38. The method of claim 22, wherein the polysorbate compound is selected from the group consisting of polysorbate 20, polysorbate 40, polysorbate 60, polysorbate 80, polysorbate 85, and mixture thereof.

39. The method of claim 27, wherein chelating agent is selected from the group consisting of ethylenediaminetetraacetic acid, pentetic acid or diethylenetriaminepentaacetic acid, N-(2-hydroxyethyl)ethylenediaminetriacetic acid, egtazic acid or ethylenebis(oxyethylenenitrilo)tetraacetic acid, nitrilotriacetic acid, dimercaptosuccinic acid, 2,3-dimercapto-1-propanol, penicillamine, and mixture thereof.

40. The method of claim 22, wherein concentrating is performed at a temperature of 4° C. for 24 hours.

41. The method of claim 22, wherein the concentrating of protein solution is performed using ultrafiltration technique that is selected from crossflow filtration technique, centrifugal filter device, and combination thereof.

42. The method of claim 22, wherein the denatured protein for regeneration is from transforming growth factor beta superfamily (TGF-b superfamily).

43. The method of claim 42, wherein the transforming growth factor beta superfamily is selected from the group consisting of bone morphogenetic protein, TGF-b, activins, growth differentiation factor (GDF), derivatives of bone morphogenetic protein, derivatives of TGF-b, derivatives of activins, derivatives of growth differentiation factor (GDF), fusions of bone morphogenetic protein, fusions of TGF-b, fusions of activins, fusions of growth differentiation factor (GDF), genetic modification of bone morphogenetic protein, genetic modification of TGF-b, genetic modification of activins, genetic modification of growth differentiation factor (GDF), and mixture thereof.
